(19) United States
(12) Patent Application Publication
(10) Pub. No.: US 2020/0352780 A1
(43) Pub. Date: Nov. 12, 2020

(54) Wearable Covering for a Lower-Extremity Immobilization Device

(71) Applicant: Jordan A. Katz, Philadelphia, PA (US)

(72) Inventor: Jordan A. Katz, Philadelphia, PA (US)

(21) Appl. No.: 16/405,549

(22) Filed: May 7, 2019

(51) Int. Cl. A61F 7/02; A61F 7/10; A61F 5/01; A61F 5/37

(52) U.S. Cl. CPC A61F 7/02; A61F 7/10; A61F 2007/0045; A61F 5/37; A61F 2007/0225; A61F 5/0111

(57) ABSTRACT

A protective covering for an immobilization device to be worn over a patient's lower extremity. The covering protects the immobilization device and underlying extremity from outside dirt, debris, and contagions, with the intention of achieving an overall improved healing rate. In various disclosed embodiments, this protective covering is either semi-permeable or impermeable to water, and includes any of a non-slip sole, pockets for holding ice packs with therapeutic effect, and a rechargeable battery.

### FIELD OF INVENTION

The disclosed covering for a lower-extremity immobilization device is to be used as a protective cover from outside dirt, debris, bacteria, mold, and other contaminants. It is intended for both indoor and outdoor use. By reducing a treated area's exposure to contaminants and the chances of additional injury through falls, this covering may improve a patient's overall healing rate. The disclosed covering may also reduce post-operative pain and swelling management with the use of built in ergonomically and location specific ice pack pockets. The disclosed covering has both a form-fitting structure and slip-resistant sole. In these ways, the covering will enhance, rather than impede, a patient's mobility during wear. Moreover, keeping the underlying boot free from dirt gathered during outdoor wear will increase compliance by patients instructed to wear the boot indoors and while sleeping.

### BACKGROUND OF THE INVENTION

Immobilization devices for the lower extremity are prescribed by medical practitioners and used by patients of various injuries. These immobilization devices include hard casts, cam boots, walking boots, pneumatic walking boots, as well as post-operative hard soled shoes. Time frame of use can be from 1 week to greater than 6-8 months. During this time period of healing, the immobilization device is exposed to significant amounts of outdoor germs and debris, which can then be tracked into the home as well as bedroom.

A need for this immobilization device cover exists to decrease influx of outdoor debris (dirt, bacteria, animal and human feces, yeast, mold, etc.) which can increase risk of disease to both the individual using the device as well as those who cohabitate with him or her.

Also, any immobilization device cover requires a slip-resistant sole to decrease further injury from slips and falls during indoor use as the entire boot must be covered, including the sole. The cover must also provide sufficient traction for use outdoors, so the slip-resistant sole must be optionally treaded. The disclosed covering is made of fabric that is optionally water resistant, water repellant, or waterproof in order to enhance durability during outdoor wear.

In addition, a need exists for a covering capable of increasing the rate of healing by decreasing post-injury and/or post-operative swelling with use of ergonomically and location-specific ice pack pockets.

Further needs for this immobilization device cover is to protect the injured individual or their significant others, family, friends from the hard plastic and sharp-edged portions of the immobilization device that can scrape or cut. These secondary injury sites may increase risk of infection if exposed to outdoor contaminants.

Other applications of the disclosed immobilization device cover are to increase aesthetics and psychological impact on the wearer, family members, friends. This improvement in aesthetics may increase compliance of immobilization device used while inside the home as well as in bed.

### SUMMARY OF THE INVENTION

The above described deficiencies are fulfilled by the device stated. The device cover will be used both indoors and outdoors as a protectant from outside germs, debris, mold, and other contaminants.

The disclosed immobilization boot cover may optionally be printed with a decorative pattern, logo, graphics, text, or advertisement.

The disclosed immobilization device cover will improve the aesthetics of the device itself which may improve compliance rate with use indoors both indoors and outdoors.

The disclosed immobilization device cover will be made of form fitting material to allow for proper fitting with decreased slippage of cover on the boot.

This form fitting immobilization device will be made in an “L” shaped manner with one end (proximal leg end) open to allow for ease of application of the immobilization device. The second end (distal foot end) will be fully enclosed in order for cover bottom of the immobilization device sole and keep outside germs/debris from indoors.

The proximal opening (leg portion) will have a closure in the form of an elastic band, Velcro strap, drawstring, or similar modality to ensure improved coverage of the proximal aspect of immobilization device to protect against transference of outside germs, debris, mold, or other contaminants into the immobilization device, the underlying treated area, or the home.

The sole of the device will be made of slip resistant material to decrease injury secondary to slipping while indoors, and to increase traction during outdoor wear. The sole will be optionally treaded to increase traction during outdoor wear.

This immobilization device cover will have a use to improve healing and reduce swelling at the treated site through the use of therapeutically located ice pack pockets. These ice packs will be situated such that the therapeutic cooling effect may be felt by the patient through both the cover and the underlying immobilization device. The disclosed pockets may be fashioned as either exterior or interior pockets, so long as they are capable of accepting a standard-sized therapeutic ice pack.

In addition, the immobilization device cover will act as a barrier from sharp edges of the boot which may come in contact with the wearer (opposite limb or other body part) or cohabitant, and lead to abrasions which may transfer germs, debris, bacteria, or other contaminants that may increase overall healing time or cause secondary injuries.

While generally described as a fabric covering for an immobilization device with a non-slip sole to be worn on a patient's lower extremity in order to reduce contamination of the treated site as well as the risk of additional injury from falls, some or all of the aspects may be implemented as a form-fitting four-way stretch Lycra covering for a cam boot with a non-slip sole and a cinched top closure, with pockets capable of holding therapeutic ice packs, and optionally a rechargeable battery, as well as other devices for performing this described functionality. The details of these and other aspects and embodiments of the present disclosure are set forth in the accompanying drawings and the description below. Other features, objects, and advantages of the disclosure will be apparent from the description and drawings, and from the claims.

### BRIEF DESCRIPTION OF THE DRAWINGS

The features, objects, and advantages of the invention will be better understood by referring to the following detailed description in conjunction with the accompanying drawings in which:

FIG. 1 illustrates a full-body view of a patient wearing the claimed protective covering with non-slip sole over the immobilization device worn on his lower extremity.

FIG. 2 illustrates the insertion of the immobilization device into the protective cover, and the insertion of therapeutic ice packs into pockets affixed to the front and back of the cover.

FIG. 3 illustrates a cross-section view of the patient's foot inside an immobilization device, which is, in turn, inside the claimed protective covering. The figure also depicts cross sections of the pockets holding ice packets to the front and back of the covering.

FIG. 4 illustrates the insertion of the immobilization device into the protective cover, and the therapeutic ice packs being inserted into interior pockets placed at the front and back of the cover.

FIG. 5 illustrates an underside view of the protective cover with a treaded, non-slip sole, Velcro flaps capable of holding ice packs at the front and back of the cover, and a pocket capable of holding a rechargeable battery.

### DETAILED DESCRIPTION OF THE DRAWINGS

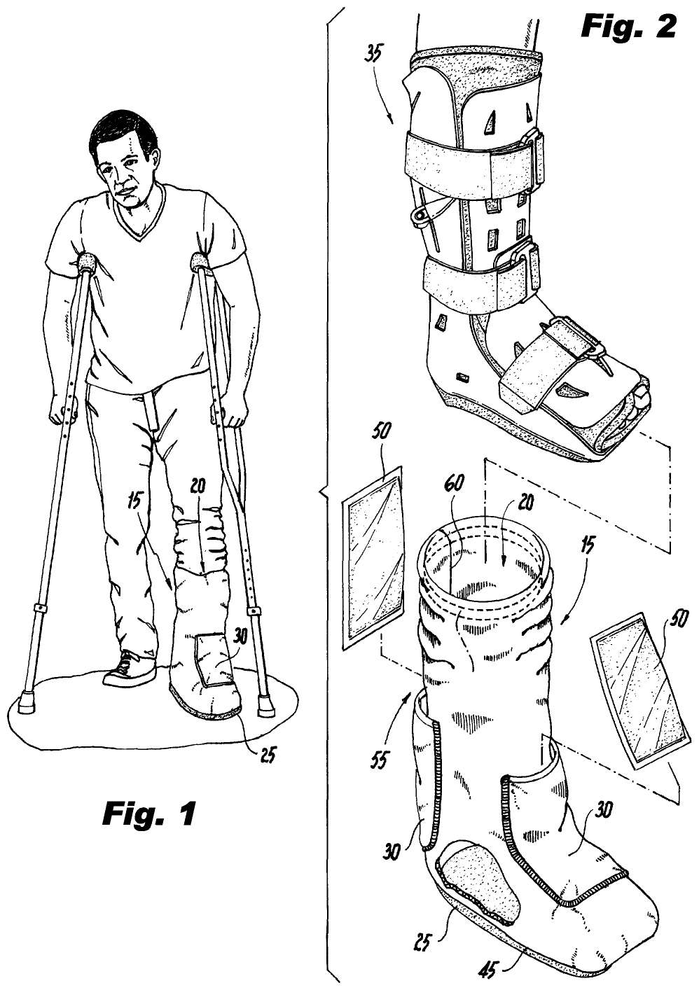

Referring to FIG. 1, an article 15 in accordance with the disclosure is illustrated. Article 15 is intended to cover an immobilization device worn on a patient's lower extremity. Such immobilization device may include, but is not limited to, a hard cast, cam boot, walking boot, pneumatic walking boot, as well as post-operative hard soled shoe. Commercial examples of immobilization devices that would be compatible with the depicted covering 15 include the Aircast FP Walking Brace, the Ossur Equalizer Walker, RCAI TA Boot, Aircast SP Walking Brace, Procare Maxtrax Air Walker, Bledsoe Low-Top Boot, Breg Controlled Range of Motion Walker, the Darco Original Med-Surg Shoe, and the DeRoyal Inline Air Walker. The items in the foregoing list are provided on an exemplary basis only, and do not represent an exhaustive list of compatible, commercially-available devices. Referring again to FIG. 1, the depicted covering 15 has a tubular, form-fitting shape with a single opening 20 at its top to permit insertion of the patient's lower extremity. The single opening 20 of the covering is cinched around the patient's leg above the top of the immobilization device, so that the entire immobilization device is covered by the article 15. In FIG. 1, the article 15 is with the claimed non-slip sole 25 joined to the covering by a single circumferential seam. FIG. 1 also depicts a pocket 30 capable of holding a therapeutic ice pack. The ice pack is located at the front of the covering 15′s sleeve, so that the therapeutic effect of the ice pack penetrates both the covering 15 and the underlying immobilization device. The pocket of FIG. 1 is an exterior pocket, as shown for exemplary purposes. As depicted in later figures, the claimed invention also contemplates interior pockets for purposes of holding at least one ice pack.

FIG. 2 depicts the insertion of an immobilization device 35 into the claimed covering 15, via the single opening 20 at the top of the tubular sleeve. The dashed line 40 around the circumference of the single opening 20 represents a band of elastic affixed to the inner surface of the opening 20. This elastic 40 cinches the sleeve's opening 20 to the patient's leg, over the top of the immobilization device 35, resulting in a hygienic closure that prevents dirt, dust, allergens, and other contaminants from penetrating the opening 20 and reaching the patient's leg beneath the immobilization device 35. This hygienic closure is intended to keep the treated anatomical area clean, thus speeding recovering time and reducing risk of infection. The tubular sleeve 55 of the claimed article 15 is fashioned from a form-fitting fabric, such as Lycra, or 4-way Stretch Lycra, and can optionally be internally reinforced with metal or plastic vertical pieces that permit the sleeve 55 to remain upright along the length of the wearer's lower extremity even when not secured by an elastic 40 at its single opening 20. FIG. 2 also depicts a non-slip sole 25 intended to aid the patient's mobility while wearing the covering 15 over his or her immobilization device 35, and decreasing the risk of further injury through slips and falls. This non-slip sole is joined to the remainder of the tubular sleeve via a single circumferential seam 45. FIG. 2 depicts dual exterior pockets 30 capable of holding therapeutic ice packs. Although the placement of these pockets in FIG. 2 to the front and back of the covering 15 is merely exemplary, the pockets of the contemplated invention should be placed such that the therapeutic cooling effect of the ice packs 50 penetrates both the fabric covering 15 and the underlying immobilization device 35. FIG. 2 also depicts insertion of the ice packs 50 into the dual exterior pockets 30 so that the ice packs 50 lay flush along the plane of the tubular sleeve 55. The tubular sleeve 55 of the claimed article 15 is fashioned from a form-fitting fabric, such as Lycra, or 4-way Stretch Lycra. The foregoing list is provided for exemplary purposes only, and is not intended to be exhaustive. Finally, FIG. 2 depicts a single seam 60 running down the back of the tubular sleeve 55. In various embodiments, this longitudinal seam can be simply sewn shut, or closed with a zipper, Velcro, snaps, or any other suitable method of closure.

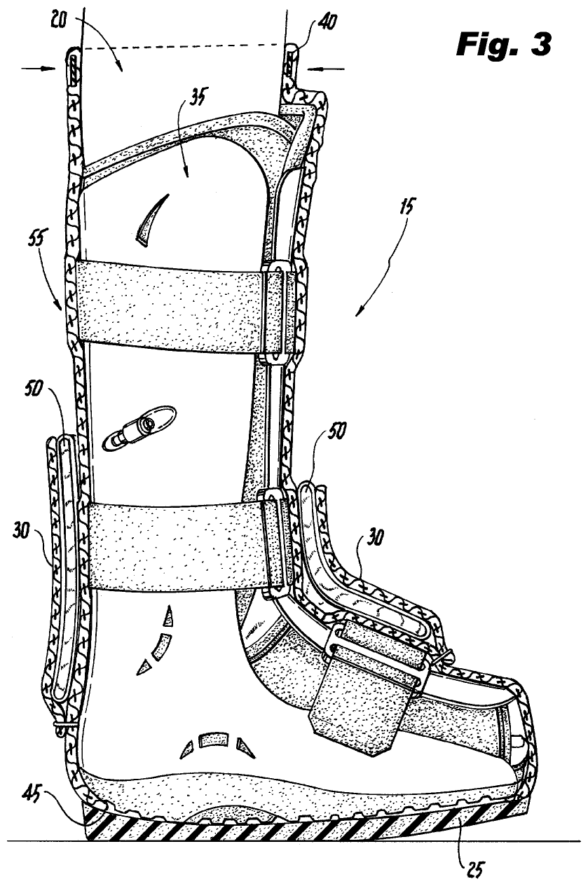

FIG. 3 depicts a cross section of the covering 15 and the underlying immobilization device 35 as worn by a patient on his or her lower extremity. The covering 15 is designed to fit snugly around the immobilization device 35, and is constructed of Lycra or 4-way stretch Lycra, or other durable, form-fitting fabrics. As in the foregoing FIGS. 1 and 2, the single opening 20 of the covering 15 cinches around the patient's leg above the upper edge of the immobilization device 35, such that the entire immobilization device 35 is covered by the article 15. FIG. 3 also depicts an elastic band 40 cinching the covering around the patient's leg such that the elastic 40 contacts the patient's limb above the top of the immobilization device 35. As discussed above, this hygienic closure is intended to keep the treated anatomical area clean, thus speeding recovering time and reducing risk of infection. FIG. 3 depicts a non-slip sole 25, designed to increase friction between the bottom of the covering 15 and the floor or ground, reducing instances of slipping and further resultant injury. The non-slip sole 25 may be constructed of rubber, fabric textile paint, or any other suitable non-slip material. The non-slip sole 25 may be flat, textured, or treaded. The non-slip sole 25 is joined to the tubular sleeve 55 of the covering 15 with a single circumferential seam 45, and the sole 25 spans the entire bottom surface of the underlying immobilization device 35. FIG. 3 also features two exterior pockets 30 as well as the underlying ice packs 50 secured in a manner flush along the length of the tubular sleeve 55. These dual exterior pockets 30 may be situated at any location along the length of the tubular sleeve 55, but are optimally located such that the therapeutic cooling effect of the ice packs 50 penetrates both the fabric covering 15 and the underlying immobilization device 35.

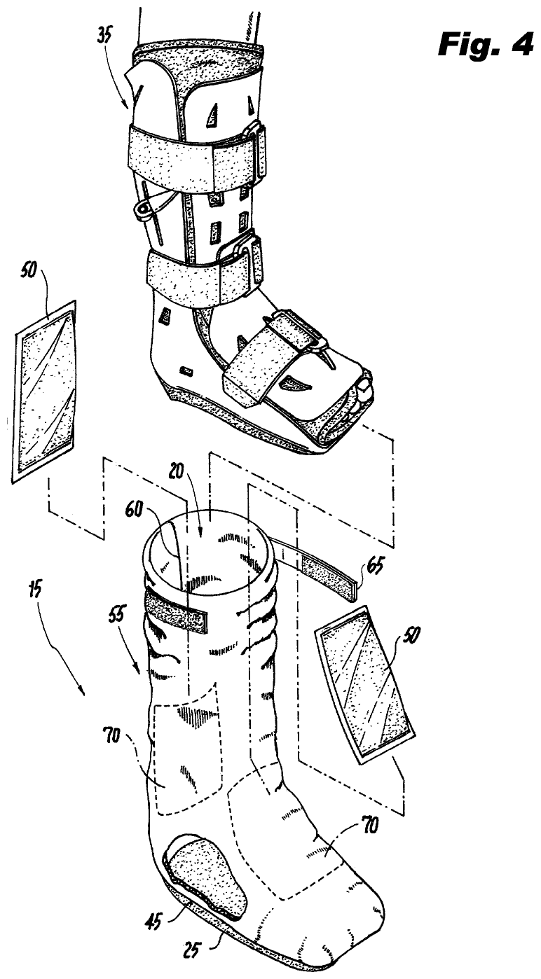

FIG. 4 depicts the insertion of an immobilization device 35 into the claimed covering 15, via the single opening 20 at the top of the tubular sleeve. In this depicted embodiment, the top of the covering 15 is cinched around the patient's leg above the upper rim of the immobilization device 35 with a Velcro strap 65 that may be adjusted to the circumference of a specific patient's leg. As with the elastic closure featured in FIGS. 2 and 3, the Velcro strap 65 results in a hygienic closure that prevents dirt, dust, allergens, and other contaminants from penetrating the closure is intended to keep the treated anatomical area clean, thus speeding recovering time and reducing risk of infection. FIG. 4 also depicts a non-slip sole 25 intended to aid the patient's mobility while wearing the covering 15 over his or her immobilization device 35, and decreasing the risk of further injury through slips and falls. This non-slip sole is joined to the remainder of the tubular sleeve via a single circumferential seam 45. FIG. 2 depicts dual interior pockets 70 capable of holding therapeutic ice packs 50 flush along the plane of the tubular sleeve 55. These interior pockets 70 are sewn into the covering 15 so that only their top opening is visible, thus shielding the exterior of the pocket from the elements and increasing lifespan of the pockets' stitching. Although the placement of these pockets in FIG. 4 to the front and back of the covering 15 is merely exemplary, the pockets of the contemplated invention should be placed such that the therapeutic cooling effect of the ice packs 50 penetrates both the fabric covering 15 and the underlying immobilization device 35. FIG. 4 also depicts insertion of the ice packs 50 into the dual interior pockets 70. The tubular sleeve 55 of the claimed article 15 is fashioned from a form-fitting fabric, such as Lycra, or 4-way Stretch Lycra, and can optionally be internally reinforced with metal or plastic vertical pieces that permit the sleeve 55 to remain upright along the length of the wearer's lower extremity even when not secured by an elastic 40, Velcro strap 65, or drawstring 80 at its single opening 20. The foregoing list is provided for exemplary purposes only, and is not intended to be exhaustive. Finally, FIG. 4 depicts a single seam 60 running down the back of the tubular sleeve 55. In various embodiments, this longitudinal seam can be simply sewn shut, or closed with a zipper, Velcro, snaps, or any other suitable method of closure.

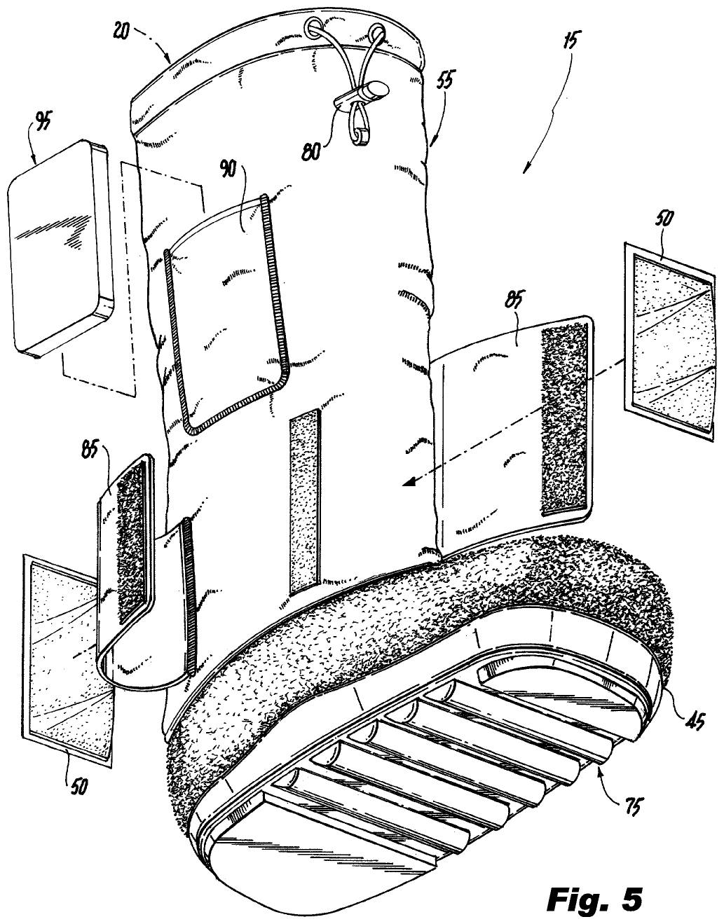

FIG. 5 depicts alternative, equally suitable embodiments of the claimed article 15. Specifically, FIG. 5 depicts the claimed covering 15 with a thicker, treaded non-slip sole 75 to increase the wearer's traction on outdoor surfaces and reduce the likelihood of further injury through falling. The treaded non-slip sole 75 is intended to increase both durability and versatility of the covering 15, and can be comprised of any suitable non-slip material, such as rubber or fabric textile paint. As in the foregoing figures, the treaded non-slip sole 75 is secured to the tubular sleeve 55 via a single circumferential seam 45. FIG. 5 also depicts a drawstring 80 as an alternate, equally viable embodiment for the closure of the covering's 15 single opening 20, which cinches around the wearer's leg at a point above the upper rim of the underlying immobilization device 35 such that the entire immobilization device 35 is contained within the tubular sleeve 55 of the covering 15. Like the elastic band 40 of FIGS. 2 and 3 and the adjustable Velcro strap 65 of FIG. 4, the drawstring 80 embedded within the circumference of the tubular sleeve's 55 single opening 20 is intended to be adjustable around the circumference of the wearer's leg. Likewise, the drawstring 80 is exemplary of any mode of cinching the tubular sleeve 55 in order to achieve a hygienic closure that prevents dirt, dust, allergens, and other contaminants from penetrating the opening 20 and reaching the patient's leg beneath the immobilization device 35. As above, the tubular sleeve 55 of the claimed article 15 is fashioned from a form-fitting fabric, such as Lycra, or 4-way Stretch Lycra, and can optionally be internally reinforced with metal or plastic vertical pieces that permit the sleeve 55 to remain upright along the length of the wearer's lower extremity even when not secured by an elastic 40, Velcro strap 65, or drawstring 80 at its single opening 20. As the claimed invention contemplates any mode of securing at least one ice pack 50 to the tubular sleeve 55 in such a way that the therapeutic cooling effect of the ice packs 50 penetrates both the fabric covering 15 and the underlying immobilization device 35, FIG. 5 depicts dual Velcro flaps 85 for securing ice packs 50 to the outside of the tubular sleeve 55. These flaps 85, like the aforementioned interior 70 and exterior 30 pockets, are acceptable for securing the ice packs 50 so that they are flush along the plane of the tubular sleeve 55. Finally, FIG. 5 depicts an additional exterior pocket 90 capable of holding a rechargeable battery 95 for the wearer's convenience. This battery 95 may be used for myriad purposes, such as charging personal electronics or monitors.

Various modifications can be made within the spirit of this disclosure and, in some cases, features described in different implementations can be combined in a single implementation. Accordingly, other implementations are within the scope of the claims.

What is claimed is:

1) A covering for an immobilization device to be worn over a patient's lower extremity, comprising:
a. a fabric sleeve with a single opening and a single longitudinal seam that extends past the top of the immobilization device;
b. a non-slip sole joined to the bottom of the fabric sleeve by a single circumferential seam;
c. a means for attaching at least one ice pack to the sleeve such that the cooling effect is felt by the wearer through both the sleeve and the underlying immobilization device; and
d. a means for cinching the single opening of the sleeve around the wearer's shin, above the top of the immobilization device.

2) The covering of claim 1, wherein the fabric is Lycra.

3) The covering of claim 2, wherein the Lycra is 4-way stretch Lycra.

4) The covering of claim 1, wherein the non-slip sole is a treaded sole.

5) The covering of claim 1, wherein the non-slip sole is comprised of rubber.

6) The covering of claim 1, wherein the non-slip sole is treated with fabric textile paint.

7) The covering of claim 1, further comprising a pocket for holding a rechargeable battery pack.

8) The covering of claim 1, wherein the fabric is treated to be water-resistant.

9) The covering of claim 1, wherein the fabric is treated to be water-repellant.

10) The covering of claim 1, wherein the fabric is treated to be waterproof.

11) The covering of claim 1, wherein the fabric sleeve is internally reinforced to stand upright when the means for cinching is not secured around the wearer's shin.

12) A covering for an immobilization device to be worn over a patient's lower extremity, comprising:
a. a fabric sleeve with a single opening and a single longitudinal seam;
b. a non-slip sole joined to the bottom of the fabric sleeve by a single circumferential seam; and
c. a pocket for attaching at least one ice pack to the sleeve such that the cooling effect is felt by the wearer through both the sleeve and the underlying immobilization device;
wherein the upper portion of the fabric sleeve extends past the top of the immobilization device, and wherein the single opening is cinched around the wearer's shin above the top of the immobilization device.

13) The covering of claim 12, wherein the fabric is Lycra.

14) The covering of claim 13, wherein the Lycra is 4-way stretch Lycra.

15) The covering of claim 12, wherein the non-slip sole is a treaded sole.

16) The covering of claim 12, wherein the non-slip sole is comprised of rubber.

17) The covering of claim 12, wherein the non-slip sole is treated with fabric textile paint.

18) The covering of claim 12, further comprising a pocket for holding a rechargeable battery pack.

19) The covering of claim 12, wherein the single opening of the fabric sleeve is cinched around the wearer's shin with an elastic joined circumferentially to the upper opening of the sleeve.

20) The covering of claim 12, wherein the single opening of the fabric sleeve is cinched around the wearer's shin with a drawstring joined circumferentially to the upper opening of the sleeve.

21) The covering of claim 12, wherein the single opening of the fabric sleeve is cinched around the wearer's shin with a Velcro strap joined circumferentially to the upper opening of the sleeve.

22) The covering of claim 12, wherein the pocket for attaching at least one ice pack to the sleeve is an interior pocket.

23) The covering of claim 12, wherein the fabric is treated to be water-resistant.

24) The covering of claim 12, wherein the fabric is treated to be water-repellant.

25) The covering of claim 12, wherein the fabric is treated to be waterproof.

26) The covering of claim 12, wherein the fabric sleeve is internally reinforced to stand upright when the means for cinching is not secured around the wearer's shin.

27) A covering for an immobilization device to be worn over a patient's lower extremity, comprising:
a. a fabric sleeve with a single opening and a single longitudinal seam;
b. a non-slip sole joined to the bottom of the fabric sleeve by a single circumferential seam; and
c. a flap for attaching at least one ice pack to the sleeve such that the cooling effect is felt by the wearer through both the sleeve and the underlying immobilization device;
wherein the upper portion of the fabric sleeve extends past the top of the immobilization device, and wherein the single opening is cinched around the wearer's shin above the top of the immobilization device.

28) The covering of claim 27, wherein the flap is secured by Velcro, at least one button, or at least one snap.

29) The covering of claim 27, wherein the fabric is Lycra.

30) The covering of claim 29, wherein the Lycra is 4-way stretch Lycra.

31) The covering of claim 27, wherein the non-slip sole is a treaded sole.

32) The covering of claim 27, wherein the non-slip sole is treated with fabric textile paint.

33) The covering of claim 27, wherein the non-slip sole is comprised of rubber.

34) The covering of claim 27, further comprising a pocket for holding a rechargeable battery pack.

35) The covering of claim 27, wherein the single opening of the fabric sleeve is cinched around the wearer's shin with an elastic joined circumferentially to the upper opening of the sleeve.

36) The covering of claim 27, wherein the single opening of the fabric sleeve is cinched around the wearer's shin with a drawstring joined circumferentially to the upper opening of the sleeve.

37) The covering of claim 27, wherein the single opening of the fabric sleeve is cinched around the wearer's shin with a Velcro strap joined circumferentially to the upper opening of the sleeve.

38) The covering of claim 27, wherein the fabric is treated to be water-resistant.

39) The covering of claim 27, wherein the fabric is treated to be water-repellant.

40) The covering of claim 27, wherein the fabric is treated to be waterproof.

41) The covering of claim 27, wherein the fabric sleeve is internally reinforced to stand upright when the means for cinching is not secured around the wearer's shin.
